(19) United States
(12) Patent Application Publication
(10) Pub. No.: US 2007/0117135 A1
(43) Pub. Date: May 24, 2007

(54) Method of inoculation for evaluating candidate therapies for prevention of acquisition of infectious diseases

(76) Inventor: John A. Willimann, Coral Gables, FL (US)

Correspondence Address: ROBERT M. DOWNEY, P.A., 6751 N. FEDERAL HWY., SUITE 300, BOCA RATON, FL, 33487, US

(21) Appl. No.: 11/604,015

(22) Filed: Nov. 22, 2006

Related U.S. Application Data
(60) Provisional application No. 60/739,987, filed on Nov. 22, 2005.

(51) Int. Cl. C12Q 1/68; C12P 21/06

(52) U.S. Cl. 435/6; 435/69.1

(57) ABSTRACT

A method of inoculating the vestibular region of the nares with virus, bacteria, fungus, or mold to ascertain whether acquisition and/or prevention of transmission of infection may occur.

This non-provisional patent application is based on provisional patent application Ser. No. 60/739,989 filed on Nov. 22, 2005.

### BACKGROUND OF THE INVENTION

1. Field of the Invention

The invention relates to inoculation with infectious organisms and more particularly, to a method for inoculating the vestibular region of the nares to evaluate candidate therapies for prevention of acquisition of infectious diseases.

2. Discussion of the Related Art

The most common mechanism of person-to-person spread of infection is direct contact followed by self-inoculation. The nose is believed to be the primary incubator of various pathogenic organisms including bacteria, virus, and mold. Infection with bacteria and virus often requires that the virus reach the pseudostratified columnar epithelium of the nasal cavity. However, the medical research community has failed to recognize that inoculation of bacteria, virus, or fungi to the vestibular region of the nose may play a major role in whether and the degree to which, acquisition of infection may occur. Instead, studies of self-inoculation have generally included intentional inoculation of the eyes with transfer of virus to the ciliated epithelium via the tear duct and/or an attempt to reach the area of the ciliated epithelium via the nares.

The anterior portion of the nose, the vestibule, is lined with keratinized squamous epithelium. This keratinized epithelium transitions posteriorly to non-keratinized squamous epithelium and then to the columnar epithelium of the nasal cavity.

Recent efforts to prevent infection have revolved around attempts to interrupt direct contact transmission by inactivating the pathogen before it reaches the pseudocolumnar epithelium. These attempts have included inactivation of the virus on the hands, or prevention of attachment of virus to the respiratory epithelium. The normal ciliary clearance of foreign material from the nose poses a formidable barrier to the use of the intranasal strategies. Inactivation of bacteria, virus, and mold in the vestibule, where ciliary clearance is not an issue, provides a strategy to overcome this barrier. The potential utility of this strategy requires an assessment of whether a virus, bacteria, fungus, or mold inoculated onto the vestibular epithelium actually contributes to the transmission of infection.

It was not known until now, under the method of the present invention, that more superficial inoculation of bacteria or virus onto the epithelium of the vestibule will result in infection.

### OBJECTS AND ADVANTAGES

Considering the foregoing, it is a primary object of the present invention to provide a method whereby the transmission and acquisition of a pathogenic organism could be observed after self-inoculation of a pathogenic organism to the vestibular region of the nares.

### SUMMARY OF THE INVENTION

The invention is directed to a method of inoculating the vestibular region of the nares with virus, bacteria, fungus, or mold to ascertain whether acquisition and/or prevention of transmission of infection may occur.

### BRIEF DESCRIPTION OF THE DRAWINGS

For a fuller understanding of the nature of the present invention, reference should be made to the following detailed description taken in conjunction with the accompanying drawings in which:

FIG. 1 is a general diagram of the nasal and sinus cavities; and

FIG. 2 is an isolated view of the anterior nares taken from the area indicated as 2 in FIG. 1.

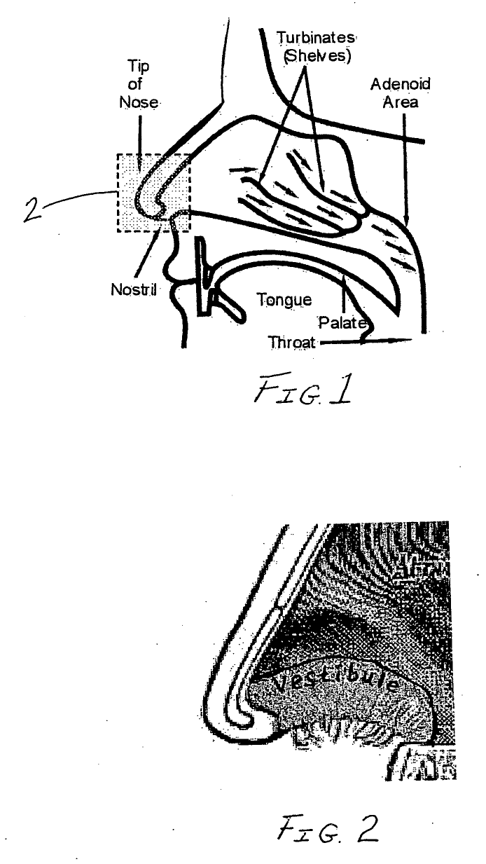

### DETAILED OF THE PREFERRED EMBODIMENT

Healthy volunteers who were found to have serum neutralizing antibody titers of <1:4 to rhinovirus type 39 were enrolled to validate the viability of the method described herein.

The challenge virus used in this study was a safety tested pool of rhinovirus type 39 (RV39, VT-51, 488772-042105). This pool has a starting titer of approximately 103.8 TCID50/mL.

Virus challenge: On the day of the virus challenge, each volunteer had a symptom score evaluated in an interactive interview with the study coordinator to assure that all were asymptomatic and had a blood specimen collected for serologic testing. Each volunteer then had approximately 160 TCID50 of RV39, contained in a volume of 10 μl, placed into the “cup” formed by the thumb and first two fingers of the right hand. The volunteers were instructed to spread the virus over the fingertips with the thumb of the right hand. When the virus challenge had dried (˜10 min), each volunteer intentionally inoculated the anterior nares with the first and second fingers of the right hand. This procedure was carefully monitored to ensure that the finger was inserted only approximately 1 cm into the nose to limit inoculation to the vestibule region of the nares, as seen in FIG. 2.

Subjects returned to the study site daily for 5 days after the virus challenge for collection of a nasal lavage specimen for viral culture. Nasal lavage was mixed 1:4 with 4× concentrated viral collecting broth and then stored frozen until cultured. Each specimen was cultured in two tube cultures of human embryonic lung fibroblast cells (one tube of MRC-5 and one tube of WI-38). These cultures were incubated on roller drums at 33° C. and observed for 10 days for development of viral cytopathic effect typical of rhinovirus. Rhinovirus isolates from subjects who did not have a serum neutralizing antibody response were neutralized with antibody to RV39 to confirm that the infection was due to the challenge serotype. Serum collected prior to challenge and again approximately 18 days later was assayed for antibody to RV39 by a microtiter neutralization assay. Volunteers with rhinovirus detected in any post-challenge culture or with at least four-fold rise in serum neutralizing antibody titer between the acute and convalescent specimens were considered infected.

Fifty percent (50%) of the volunteers challenged with RV39 in this study became infected with the challenge virus (95% CI: 0.24-0.76). Three volunteers had both virus isolation seroconversion, two volunteers had infection documented by virus isolation alone.

Conclusions: Inoculation of the vestibule of the nares resulted in infection of 50% of challenged subjects in this study. These results document the feasibility of this route of infection and suggest that inactivation of virus by virucidal treatment of the nasal vestibule will potentially have an impact on rhinovirus infections transmitted by direct contact.

What is claimed is:

1. A method for evaluating candidate therapies for the prevention of acquisition of infectious diseases, comprising the steps of:
selecting at least one test pathogen from the group consisting of virus, bacteria, fungus and mold;
applying said test pathogen to the vestibular region of the nares of a test subject; and
subsequently determining whether the test subject has been infected with the test pathogen.

2. The method as recited in claim 1 wherein said step of subsequently determining whether the test subject has been infected with the test pathogen includes the further step of:
collecting a specimen from the nares of the test subject for culture.

3. The method as recited in claim 1 further comprising of the step of:
selecting the test subject based on the test subject's level of antibody to the test pathogen prior to said step of applying said test pathogen to the vestibular region of the nares of the test subject.

4. The method as recited in claim 3 wherein said step of subsequently determining whether the test subject's has been infected with the test pathogen includes the further step of:
measuring the level of antibody to the test pathogen in the test subject to determine the test subject neutralizing antibody response to the test pathogen.

5. A method for evaluating candidate therapies for the prevention of acquisition of infectious diseases, comprising the steps of:
selecting a test pathogen;
applying said test pathogen to the vestibular region of the nares of a test subject; and
subsequently determining whether the test subject has been infected with the test pathogen.

6. The method as recited in claim 5 wherein said step of subsequently determining whether the test subject has been infected with the test pathogen includes the further step of:
collecting a specimen from the nares of the test subject for culture.

7. A method for ascertaining whether acquisition of infection or prevention of transmission of infection with a pathogen may occur among humans, said method comprising the steps of:
selecting a test pathogen from the group consisting of virus, bacteria, fungus and mold;
applying said test pathogen to the vestibular region of the nares of a mammal; and
subsequently determining whether the mammal has been infected with the test pathogen.
